(19) United States
(12) Patent Application Publication
(10) Pub. No.: US 2002/0077355 A1
(43) Pub. Date: Jun. 20, 2002

(54) Use of epoxyeicosatrienoic acids in the treatment of cerebrovascular conditions

(76) Inventors: James K. Liao, Weston, MA (US); Michael A. Moskowitz, Belmont, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 09/870,425

(22) Filed: May 30, 2001

Related U.S. Application Data
(60) Provisional application No. 60/207,978, filed on May 30, 2000.

(51) Int. Cl. A61K 31/336; A61K 31/20

(52) U.S. Cl. 514/475; 514/560

(57) ABSTRACT

The invention relates to the use of epoxyeicosatrienoic acids and/or inducers of cytochrome P-450 epoxygenase activity to reduce brain injury in a subject with a cerebrovascular condition, including stroke.

### RELATED APPLICATIONS

This application claims priority under 35 USC § 119(e) from U.S. Provisional Patent Application Ser. No. 60/207,978 filed on May 30, 2000, entitled USE OF EPOXYEICOSATRIENOIC ACIDS IN THE TREATMENT OF CEREBROVASCULAR CONDITIONS. The contents of the provisional application are hereby expressly incorporated by reference.

### FIELD OF THE INVENTION

This invention relates to the use of epoxyeicosatrienoic acids and/or inducers of cytochrome P-450 epoxygenase activity to reduce brain injury in a subject with a cerebrovascular condition, including stroke.

### BACKGROUND OF THE INVENTION

Cerebrovascular conditions may be caused by one of several pathologic processes involving blood vessels in the brain. The process may be: (i) intrinsic to the vessel (e.g., atherosclerosis), (ii) originate remotely (e.g., when an embolus from the heart lodges in an intracranial vessel), (iii) result from decreased perfusion pessure or increased blood viscosity with inadequate cerebral blood flow, or (iv) result from rupture of a vessel in the subarachnoid space or intracerebrla tissue (Harrison's Principles of Experimental Medicine, 13th Edition, McGraw-Hill, Inc. New York, p. 2233).

Cerebrovascular conditions include stroke, subarachnoid hemorhage, traumatic brain injury, and CADACIL syndrome.

Stroke is often cited as the third most common cause of death in the industrial world, ranking behind ischemic heart disease and cancer. Strokes are responsible for about 300,000 deaths annually in the United States and are a leading cause of hospital admissions and long-term disabilities. Accordingly, the socioeconomic impact of stroke and its attendant burden on society is practically immeasurable.

“Stroke” is defined by the World Health Organization as a rapidly developing clinical sign of focal or global disturbance of cerebral function with symptoms lasting at least 24 hours. Strokes are also implicated in deaths where there is no apparent cause other than an effect of vascular origin.

Strokes are typically caused by blockages or occlusions of the blood vessels to the brain or within the brain. With complete occlusion, arrest of cerebral circulation causes cessation of neuronal electrical activity within seconds. Within a few minutes after the deterioration of the energy state and ion homeostasis, depletion of high energy phosphates, membrane ion pump failure, efflux of cellular potassium, influx of sodium chloride and water, and membrane depolarization occur. If the occlusion persists for more than five to ten minutes, irreversible damage results. With incomplete ischemia, however, the outcome is difficult to evaluate and depends largely on residual perfusion and the availability of oxygen. After a thrombotic occlusion of a cerebral vessel, ischemia is rarely total. Some residual perfusion usually persists in the ischemic area, depending on collateral blood flow and local perfusion pressure.

Cerebral blood flow can compensate for drops in mean arterial blood pressure from 90 to 60 mm Hg by autoregulation. This phenomenon involves dilatation of downstream resistant vessels. Below the lower level of autoregulation (about 60 mm Hg), vasodilatation is inadequate and the cerebral blood flow falls. The brain, however, has perfusion reserves that can compensate for the fall in cerebral blood flow. This reserve exists because under normal conditions only about 35% of the oxygen delivered by the blood is extracted. Therefore, increased oxygen extraction can take place, provided that normoxia and normocapnea exist. When distal blood pressure falls below approximately 30 mm Hg, the two compensatory mechanisms (autoregulation and perfusion reserve) are inadequate to prevent failure of oxygen delivery.

As blood flow drops below the ischemic threshold of 23 ml/100 g/minute, symptoms of tissue hypoxia develop. Severe ischemia may be lethal. When the ischemia is moderate, it will result in “penumbra.” In the neurological context, penumbra refers to a zone of brain tissue with moderate ischemia and paralyzed neuronal function, which is reversible with restoration of adequate perfusion. The penumbra forms a zone of collaterally perfused tissue surrounding a core of severe ischemia in which an infarct has developed. In other words, the penumbra is the tissue area that can be saved, and is essentially in a state between life and death.

Although an ischemic event can occur anywhere in the vascular system, the carotid artery bifurcation and the origin of the internal carotid artery are the most frequent sites for thrombotic occlusions of cerebral blood vessels, which result in cerebral ischemia. The symptoms of reduced blood flow due to stenosis or thrombosis are similar to those caused by middle cerebral artery disease. Flow through the ophthalmic artery is often affected sufficiently to produce amaurosis fugax or transient monocular blindness. Severe bilateral internal carotid artery stenosis may result in cerebral hemispheric hypoperfusion. This manifests with acute headache ipsilateral to the acutely ischemic hemisphere. Occlusions or decrease of the blood flow with resulting ischemia of one anterior cerebral artery distal to the anterior communicating artery produces motor and cortical sensory symptoms in the contralateral leg and, less often, proximal arm. Other manifestations of occlusions or underperfusion of the anterior cerebral artery include gait ataxia and sometimes urinary incontinence due to damage to the parasagittal frontal lobe. Language disturbances manifested as decreased spontaneous speech may accompany generalized depression of psychomotor activity.

Most ischemic strokes involve portions or all of the territory of the middle cerebral artery with emboli from the heart or extracranial carotid arteries accounting for most cases. Emboli may occlude the main stem of the middle cerebral artery, but more frequently produce distal occlusion of either the superior or the inferior branch. Occlusions of the superior branch cause weakness and sensory loss that are greatest in the face and arm. Occlusions of the posterior cerebral artery distal to its penetrating branches cause complete contralateral loss of vision. Difficulty in reading (dyslexia) and in performing calculations (dyscalculia) may follow ischemia of the dominant posterior cerebral artery. Proximal occlusion of the posterior cerebral artery causes ischemia of the branches penetrating to calamic and limbic structures. The clinical results are hemisensory disturbances that may chronically change to intractable pain of the defective side (thalamic pain).

Subarachnoid hemorrhage is a disorder which involves bleeding beneath the membrane covering the brain (i.e., the arachnoid). It occurs in roughly 1 in 10,000 people and is the underlying cause of approximately 5-10% of strokes. Subarachnoid hemorrhage in turn can lead to cerebral vasospasms (i.e., constriction of a blood vessel) for which there are no single effective drugs.

Subjects having a subarachnoid hemorrhage can be identified by a number of symptoms. For example, a subject having a subarachnoid hemorrhage will present with blood in the subarachnoid, usually in a large amount. Subjects having a subarachnoid hemorrhage can also be identified by an intracranial pressure that approximates mean arterial pressure, by a fall in cerebral perfusion pressure or by the sudden transient loss of consciousness (sometimes preceded by a painful headache). In about half of cases, subjects present with a severe headache which may be associated with physical exertion. Other symptoms associated with subarachnoid hemorrhage include nausea, vomiting, memory loss, hemiparesis and aphasia. Subjects having a subarachnoid hemorrhage can also be identified by the presence of creatine kinase-BB isoenzyme activity in their CSF. This enzyme is enriched in the brain but is normally not present in the CSF. Thus, its presence in the CSF is indicative of “leak” from the brain into the subarachnoid. Assay of creatine-kinase BB isoenzyme activity in the CSF is described by Coplin et al. (Coplin, et al, Arch Neurol, 1999, 56(11):1348-1352) Additionally, a spinal tap or lumbar puncture can be used to demonstrate if there is blood present in the CSF, a strong indication of a subarachnoid hemorrhage. A cranial CT scan or an MRI can also be used to identify blood in the subarachnoid region. Angiography can also be used to determine not only whether a hemorrhage has occurred but also the location of the hemorrhage.

Subarachnoid hemorrhage commonly results from rupture of an intracranial saccular aneurysm or from malformation of the arteriovenous system in, and leading to, the brain. Accordingly, a subject at risk of having a subarachnoid hemorrhage includes subjects having a saccular aneurysm as well as subjects having a malformation of the arteriovenous system. It is estimated that 5% of the population have such aneurysms yet only 1 in 10,000 people actually have a subarachnoid hemorrhage. The top of the basilar artery and the junction of the basilar artery with the superior cerebellar or the anterior inferior cerebellar artery are common sites of saccular aneurysms. Subjects having a subarachnoid hemorrhage may be identified by an eye examination, whereby slowed eye movement may indicate brain damage. A subject with a developing saccular aneurysm can be identified through routine medical imaging techniques, such as CT and MRI. A developing aneurysm forms a mushroom-like shape (sometimes referred to as “a dome with a neck” shape). One of the most common causes of subarachnoid hemorrhage is traumatic brain injury.

Traumatic brain injury is a major cause of disability and is the leading source of brain damage in previously healthy adults in the United States. Motor vehicle accidents account for nearly 50% of all traumatic brain injuries. The second leading cause of traumatic head injury in the United States is firearm related injuries. Falls account for a large proportion of non-fatal traumatic head injuries. Ten million people in the United States suffer head injuries yearly, of which 500,000 require hospitalization.

Cerebral autosomal dominant arteriopathy with subcortical infarcts and leucoencephalopathy (CADASIL) syndrome, is a disorder characterized by relapsing strokes with neuropsychiatric symptoms that affects relatively young adults of both sexes. CT scans have demonstrated occlusive cerebrovascular infarcts in the white matter, which is usually reduced.

The eventual extent of neurologic recovery from a cerebral infarction in a cerebrovascular condition depends on the patient's age and general state of health as well as on the site and size of the infarction. Impaired consciousness, mental deterioration, aphasia, or severe brainstem signs all suggest a poor prognosis. Complete recovery is uncommon, but the sooner improvement begins, the better the prognosis. About 50% of patients with moderate or severe hemiplegia, and most of those with lesser deficits, recover functionally by the time of discharge and are ultimately able to care for their basic needs, have a clear sensorium, and can walk adequately, although use of an affected limb may be limited. Any deficit remaining after 6 months is likely to be permanent, although some patients continue to improve slowly. Recurrence of cerebral infarction is relatively common, and each recurrence is likely to add to the neurologic disability.

Because of the debilitating effects of cerebrovascular conditions, there continues to exist a need for effective treatments.

### SUMMARY OF THE INVENTION

The invention involves the discovery that EETs and/or inducers of cytochrome P-450 epoxygenase activity reduce brain injury in a subject with a cerebrovascular condition. More specifically, it has been discovered that EETs and/or inducers of cytochrome P-450 epoxygenase activity are useful in reducing brain injury caused by a cerebrovascular condition in a subject.

It has been discovered, unexpectedly, that acute administration of EETs and/or inducers of cytochrome P-450 epoxygenase activity to subjects with a cerebrovascular condition, is useful in reducing infarct size of an infarct caused by such condition, in some instances even by 40% (as compared to controls, e.g., subjects on placebos).

According to one aspect the invention, a method for reducing brain injury resulting from a cerebrovascular condition in a subject, is provided. The method involves administering to a subject having a cerebrovascular condition an epoxyeicosatrienoic acid, in an amount effective to reduce brain injury in the subject. In certain embodiments, the cerebrovascular condition is selected from the group consisting of stroke, subarachnoid hemorhage, traumatic brain injury, and CADACIL syndrome. The epoxyeicosatrienoic acid can be in any of its isomeric forms. In some embodiments, epoxyeicosatrienoic acid isomers include 5,6-epoxyeicosatrienoic acid, 8,9-epoxyeicosatrienoic acid, 11,12 -epoxyeicosatrienoic acid, and 14,15-epoxyeicosatrienoic acid. In important embodiments, the epoxyeicosatrienoic acid is 11,12-epoxyeicosatrienoic acid. In certain embodiments, the method further comprises co-administering arachidonic acid, a cytochrome P-450 arachidonic acid epoxygenase inducer, or a combination thereof. Cytochrome P-450 arachidonic acid epoxygenase inducers are as described below.

According to another aspect of the invention, a method for reducing brain injury resulting from a cerebrovascular condition in a subject using a cytochrome P-450 arachidonic acid epoxygenase inducer, is provided. The method involves administering to a subject having a cerebrovascular condition, a cytochrome P-450 arachidonic acid epoxygenase inducer in an amount effective to reduce brain injury in the subject. In certain embodiments, the cerebrovascular condition is selected from the group consisting of stroke, subarachnoid hemorhage, traumatic brain injury, and CADACIL syndrome. In some embodiments, the cytochrome P-450 arachidonic acid epoxygenase inducer includes 2,3,7,8-tetrachlorodibenzo-p-dioxin, β-naphthoflavone, 3-methylcholanthrene, or a combination thereof. In further embodiments, the method may further comprise co-administering arachidonic acid, an epoxyeicosatrienoic acid, or a combination thereof.

The invention also involves the co-administration of agents known to be beneficial in reducing brain injury in a cerebrovascular condition according to any of the foregoing aspects and embodiments of the invention. In certain embodiments, agents known to be beneficial in reducing brain injury in a cerebrovascular condition include anticoagulants, antiplatelet drugs, HMG-CoA reductase inhibitors, and/or agents that upregulate endothelial cell nitric oxide synthase.

These and other aspects of the invention, as well as various advantages and utilities, will be more apparent with reference to the detailed description of the preferred embodiments.

### BRIEF DESCRIPTION OF THE DRAWINGS

FIG. 1 is a bar graph showing the effects of EET and vehicle infusion in animals which have experienced focal cerebral ischemia.

FIG. 2 is a graph showing the effects of EET and vehicle infusion to the cerebral blood flow (CBF) in animals which have experienced focal cerebral ischemia over time; CBF is represented as a % of CBF over baseline.

FIG. 3 is a graph showing the effects of EET and vehicle infusion to the mean arterial blood pressure (MABP) of animals which have experienced focal cerebral ischemia over time.

### DETAILED DESCRIPTION OF THE INVENTION

The invention involves the discovery that EETs and/or inducers of cytochrome P-450 epoxygenase activity reduce brain injury in a subject with a cerebrovascular condition. More specifically, it has been discovered that EETs and/or inducers of cytochrome P-450 epoxygenase activity are useful in reducing brain injury caused by a cerebrovascular condition in a subject.

It has been discovered, unexpectedly, that administration of EETs and/or inducers of cytochrome P-450 epoxygenase activity to subjects with a cerebrovascular condition, is useful in reducing infarct size of an infarct caused by such condition, in some instances even by 40% (as compared to controls, e.g., subjects on placebos).

A subject as used herein includes humans, non human primates, dogs, cats, sheep, goats, cows, pigs, horses and rodents. The invention thus is useful for therapeutic purposes and also is useful for research purposes such as in testing in animal or in vitro models of medical, physiological or metabolic pathways or conditions.

The invention provides methods for the treatment of a cerebrovascular condition. According to the present invention, EETs and/or inducers of cytochrome P-450 epoxygenase activity are administered to subjects to limit the extent of stroke-associated infarcts (i.e., to reduce brain injury).

Cytochrome P-450 epoxygenase catalyzes the NADPH-dependent epoxidation of arachidonic acid to 5,6-, 8,9-, 11,12-, and 14,15-epoxyeicosatrienoic acids (EETs), in a regio- and stereoselective manner (Capdevila, J. H., et al., FASEB J, 1992, 6:731-736).

“Epoxyeicosatrienoic acids (EETs),” as used herein, refer to all epoxyeicosatrienoic acid isomers, such as 5,6-epoxyeicosatrienoic acid, 8,9-epoxyeicosatrienoic acid, 11,12-epoxyeicosatrienoic acid, and 14,15-epoxyeicosatrienoic acid, and all of their derivatives, including their dihydroxy, salt and/or sulfonimide derivatives. A number of EETs and/or EET derivatives are readily available from commercial sources (e.g., Sigma Chemical Co., St Louis, Mo.), or they can be synthesized according to methods known in the art (e.g., EET-sulfonimides can be synthesized as described in Chen, J-K, et al., J Biol. Chem., 1998, 273:29254-29261).

“Cytochrome P-450 arachidonic acid epoxygenase inducers,” as used herein, include agents that, as the name implies, upregulate cytochrome P-450 epoxygenase activity to yield more EETs. Cytochrome P-450 arachidonic acid epoxygenase inducers include 2,3,7,8-tetrachlorodibenzo-p-dioxin, β-naphthoflavone, and 3-methylcholanthrene. Cytochrome P-450 arachidonic acid epoxygenase inducers also are readily available from commercial sources (e.g., Sigma Chemical Co., St Louis, Mo.).

The methods of the invention are advantageous for the treatment of various cerebrovascular conditions. “Cerebrovascular conditions,” as used herein, refer to stroke, subarachnoid hemorhage, traumatic brain injury, and CADACIL syndrome. A brief description for each of the foregoing cerebrovascular conditions can be fould elsewhere in the specification.

In its broadest sense, “treatment” or “to treat,” as used herein, refers to an acute treatment. “Acute treatment,” as used herein, refers to the treatment of a subject with a cerebrovascular condition by administering an agent of the invention (e.g., an EET, and/or a cytochrome P-450 epoxygenase inducer) at the onset of symptoms of the condition or at the onset of a substantial change in the symptoms of an existing condition, to the subject, in an amount effective to reduce brain injury in the subject. In most subjects, administration of the treatment preferably is begun shortly after the onset of symptoms of the condition, since early intervention will maximize the extent of potentially salvageable penumbral tissue. Treatment may be initiated, however, at any point in time prior to the completion of the infarction process, as assessed both on the basis of physical findings on neurological examination of the subject, as well as on the basis of imaging studies such as computed tomography or magnetic resonance imaging. In certain instances, the methods of the invention may be used to treat a subject after the completion of a cerebrovascular episode.

As mentioned earlier, a surprising discovery of the invention is the substantial reduction in infarct size observed (in some instances even by 40%, see Examples), of an infarct caused by a cerebrovascular condition when the subject in need of such treatment is acutely treated with an agent of the invention.

The agents of the invention used for the treatment of a cerebrovascular condition are administered in effective amounts. In general, an effective amount is any amount sufficient to reduce brain injury in a subject. Direct measures reducing or stabilizing infarct size. Other measures are well known to those of ordinary skill in the art, and include functional tests, e.g., mental and motor tests. In general, it is desired to increase EET and/or a cytochrome P-450 epoxygenase inducer levels in the brain.

In general, an effective amount is that amount of a pharmaceutical preparation that alone, or together with further doses or co-administration of other agents, produces the desired response. This can be monitored by routine methods. Generally, doses of active compounds would be from about 0.01 mg/kg per day to 1000 mg/kg per day. It is expected that doses ranging from 50-500 mg/kg will be suitable, and in one or several administrations per day.

The exact amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. It is preferred generally that a maximum dose be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The agents useful according to the invention (e.g., EETs and/or a cytochrome P-450 epoxygenase inducer) may be combined, optionally, with a pharmaceutically-acceptable carrier. The term “pharmaceutically-acceptable carrier” as used herein means one or more compatible solid or liquid fillers, diluents or encapsulating substances which are suitable for administration to a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

A variety of administration routes are available. The particular mode selected will depend, of course, upon the particular drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous preparation of reductase inhibitors, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

Other delivery systems can include time-release, delayed release or sustained release delivery systems. Such systems can avoid repeated administrations of the active compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

The invention also involves the co-administration of agents that are not EETs or inducers of cytochrome P-450 epoxygenase activity, but that can act cooperatively, additively or synergistically with such agents to reduce infarct size/brain injury associated with a cerebrovascular condition (i.e., agents that are known to be beneficial in the treatment of a cerebrovascular condition). Such agents include anticoagulants, antiplatelet agents, HMG-CoA reductase inhibitors, and agents that upregulate endothelial cell nitric oxide synthase.

Anticoagulant agents include, but are not limited to, Ancrod; Anticoagulant Citrate Dextrose Solution; Anticoagulant Citrate Phosphate Dextrose Adenine Solution; Anticoagulant Citrate Phosphate Dextrose Solution; Anticoagulant Heparin Solution; Anticoagulant Sodium Citrate Solution; Ardeparin Sodium; Bivalirudin; Bromindione; Dalteparin Sodium; Desirudin; Dicumarol; Heparin Calcium; Heparin Sodium; Lyapolate Sodium; Nafamostat Mesylate; Phenprocoumon; Tinzaparin Sodium; Warfarin Sodium.

Heparin may stabilize symptoms in evolving stroke, but anticoagulants are useless (and possibly dangerous) in acute completed stroke, and are contraindicated in hypertensives because of the increased possibility of hemorrhage into the brain or other organs. Although the timing is controversial, anticoagulants may be started to prevent recurrent cardiogenic emboli. Clot lysing agents, including tissue plasminogen activator and streptokinase, are being evaluated for the very early treatment of acute stroke. Nimodipine has recently been shown to improve survival and clinical outcome after ischemic stroke.

Other than aspirin, ticlopidine is another antiplatelet agent that has been shown to be beneficial for stroke treatment. Endarterectomy may be indicated in patients with 70 to 99 percent narrowing of a symptomatic internal carotid artery. However, most authorities agree that carotid endarterectomy is not indicated in patients with TIAs that are referable to the basilar-vertebral system, in patients with significant deficits from prior strokes, or in patients in whom a stroke is evolving.

HMG-CoA (3-hydroxy-3-methylglutaryl-coenzyme A) reductase is the microsomal enzyme that catalyzes the rate limiting reaction in cholesterol biosynthesis (HMG-CoA6Mevalonate). An HMG-CoA reductase inhibitor inhibits HMG-CoA reductase, and as a result inhibits the synthesis of cholesterol. A number of HMG-CoA reductase inhibitors has been used to treat individuals with hypercholesterolemia. More recently, HMG-CoA reductase inhibitors have been shown to be beneficial in the treatment of stroke (Endres M, et al., Proc Natl Acad Sci USA, 1998, 95:8880-5).

HMG-CoA reductase inhibitors useful for co-administration with the agents of the invention include, but are not limited to, simvastatin (U.S. Pat. No. 4, 444,784), lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5,273,995), cerivastatin, and numerous others described in U.S. Pat. Nos. 5,622,985, 5,135,935, 5,356,896, 4,920,109, 5,286,895, 5,262,435, 5,260,332, 5,317,031, 5,283,256, 5,256,689, 5,182,298, 5,369,125, 5,302,604, 5,166,171, 5,202,327, 5,276,021, 5,196,440, 5,091,386, 5,091,378, 4,904,646, 5,385,932, 5,250,435, 5,132,312, 5,130,306, 5,116,870, 5,112,857, 5,102,911, 5,098,931, 5,081,136, 5,025,000, 5,021,453, 5,017,716, 5,001,144, 5,001,128, 4,997,837, 4,996,234, 4,994,494, 4,992,429, 4,970,231, 4,968,693, 4,963,538, 4,957,940, 4,950,675, 4,946,864, 4,946,860, 4,940,800, 4,940,727, 4,939,143, 4,929,620, 4,923,861, 4,906,657, 4,906,624 and 4,897,402, the disclosures of which patents are incorporated herein by reference.

Nitric oxide (NO) has been recognized as an unusual messenger molecule with many physiologic roles, in the cardiovascular, neurologic and immune systems (Griffith, TM et al., J Am Coll Cardiol, 1988, 12:797-806). It mediates blood vessel relaxation, neurotransmission and pathogen suppression. NO is produced from the guanidino nitrogen of L-arginine by NO Synthase (Moncada, S and Higgs, E A, Eur J Clin Invest, 1991, 21:361-374). Agents that upregulate endothelial cell Nitric Oxide Synthase include, but are not limited to, L-arginine, rho GTPase function inhibitors (see International Application WO 99/47153, the disclosure of which is incorporated herein by reference), and agents that disrupt actin cytoskeletal organization (see International Application WO 00/03746, the disclosure of which is incorporated herein by reference).

“Co-administering,” as used herein, refers to administering simultaneously two or more compounds of the invention (e.g., an EET and a cytochrome P-450 epoxygenase inducer, or an EET and an agent known to be beneficial in the treatment of a cardiovascular condition—for example an anticoagulant-), as an admixture in a single composition, or sequentially, close enough in time so that the compounds may exert an additive or even synergistic effect, i.e., on reducing brain injury in a cerebrovascular condition.

The invention will be more fully understood by reference to the following examples. These examples, however, are merely intended to illustrate the embodiments of the invention and are not to be construed to limit the scope of the invention.

### EXAMPLES

Experimental Procedures

In vivo model

Preparation of Animals

Young adult male mice (C57B1/J6, Jackson Laboratories) weighing 20-25 g, were given free access to food and water prior to experimentation.

Ischemia was produced by occluding the left middle cerebral artery (MCA) with a silicon-coated 8-0 nylon monofilament under anesthesia as described (Huang, Z et al., J Cereb Blood Flow Metab, 1996, 16:981-987, Huang, Z et al., Science, 1994, 265:1883-1885, Hara, H et al., J Cereb Blood Flow Metab, 1997, 1:515-526). Arterial blood pressure, heart rate, arterial oxygen pressure, and partial pressure of carbon dioxide were monitored as described (Huang, Z et al., J Cereb Blood Flow Metab, 1996, 16:981-987, Huang, Z et al., Science, 1994, 265:1883-1885, Hara, H et al., J Cereb Blood Flow Metab, 1997, 1:515-526).

11,12 EET (Sigma Chemical Co., St Louis, Mo.) or vehicle was administered by an infusion pump (Harvard Apparatus, South Natick, Mass.) with a tethering device (Instech, Plymouth Meeting, Pa.) into the femoral vein at the rate of 0.2 ml/hour for 2 hours beginning after the onset of ischemia to deliver a dose of 30, 100, and/or 300 μg/kg/hour. Control animals received the equivalent amount of vehicle alone. During infusion, the anesthesia was discontinued. This was found to be necessary for all animals, to ensure survival during the first 24 hours after surgery. After infusion, the animals were re-anesthetized with halothane and the venous catheter was removed. The filaments were withdrawn after 2 hours and after 24 hours, and mice were sacrificed. Brains were divided into five coronal 2-mm sections using a mouse brain matrix (RBM-200C, Activated Systems, Ann Arbor, Mich., USA). Infarction volume was quantitated with an image analysis system (M4, St. Catharines, Ontario, Canada) on 2% 2,3,5-triphenyltetrazolium chloride (Sigma Chemical Co., St Louis, Mo.) stained 2-mm slices.

In a separate set of ventilated, halothane-anesthetized animals, regional cerebral blood flow (rCBF) was determined at the margins (“penumbra”) of ischemic infarcts using laser Doppler flowmetry (Perimed, PF-2B, Stockholm, Sweden) and recorded on a MacLab/8 data acquisition system (AD Instruments, Milford, Mass.). the left femoral artery and vein were cannulated for blood pressure measurement, blood gas determination and drug administration, a fiberoptic probe (Perimed PF-319:2, diameter=0.5 mm) was fixed to the skull, 4 mm lateral and 2 mm posterior to bregma on the ipsilateral hemisphere and away from large pial vessels. This coordinate identifies the site on the convex brain surface within the vascular territory supplied by the distal segment of the MCA (Huang, Z et al., Science, 1994, 265:1883-1885; Yang, G et al., Stroke, 1994, 25:165-70). rCBF was recorded continuously before, during and after MCA occlusion. The mean values after MCA occlusion and before administration were taken as baseline, and the data thereafter were expressed as percentages of the baseline value. The percent change in cerebrovascular resistance was calculated using the following formula: Δ%R=Δ% MABP/Δ% rCBF, where Δ% MABP is percent change in mean arterial blood pressure.

Data is expressed as means ±SE. Statistical evaluation was performed by Student's t-test to compare infarct volumes between groups. Analysis of variance with repeated measures followed by Dunnett's t-test was used to test the significance of rCBF and resistance changes between groups. p<0.05 was considered statistically significant.

In Vitro Cell Culture Model

Cerebellar cortices of postnatal day 7 rat pups were isolated, incubated in 0.25% trypsin for 20 min and triturated with a 10-cc Falcon pipette. Cells were plated on Falcon plastic dishes ˜100,000 cells/cm² in BME medium containing 25 mM KCl. Cell cultures were maintained in a humidified 5% CO₂ /37° C. incubator. 10 μM AraC was added from day 2-3 in vitro to suppress glial proliferation. Cells were studied at day 7-8 in vitro.

Dihydreothidine study to monitor intracellular superoxide production: 10 μl of dihydroethidine stock solution (1 mg/cc) in dimethylsulfoxide (DMSO) and 4 μl of H₂O₂ were added to 4cc of Eagle's Basal Medium (medium A). Each 2-cm² well of containing neuronal cell culture was preincubated with 11.12-EET (1.2 μM) for an hour and was replaced with 250 μL of medium A in the presence or absence of 11,12-EET (1.2 μM). Cells were examined under confocal microscopy with 488 nm laser emission and 585 nm long-pass filter. Fluorescent images of cells were obtained by photography at 10 min intervals.

Results

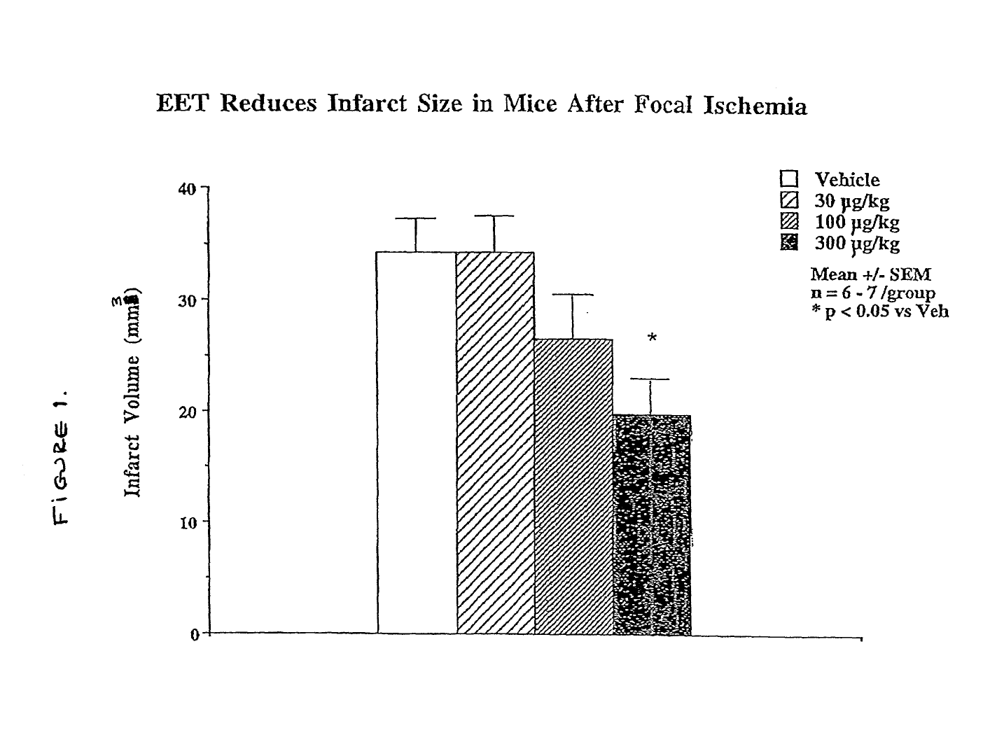

EET treatment reduced infarct volumes by at least 41% in comparison to the vehicle-treated animals (300 μg/kg group) (20±3 vs. 34±3 of hemispheric volume, respectively, p<0.05, n=7 for each group, * denotes statistically significant difference p<0.05) (Table 1 and FIG. 1).

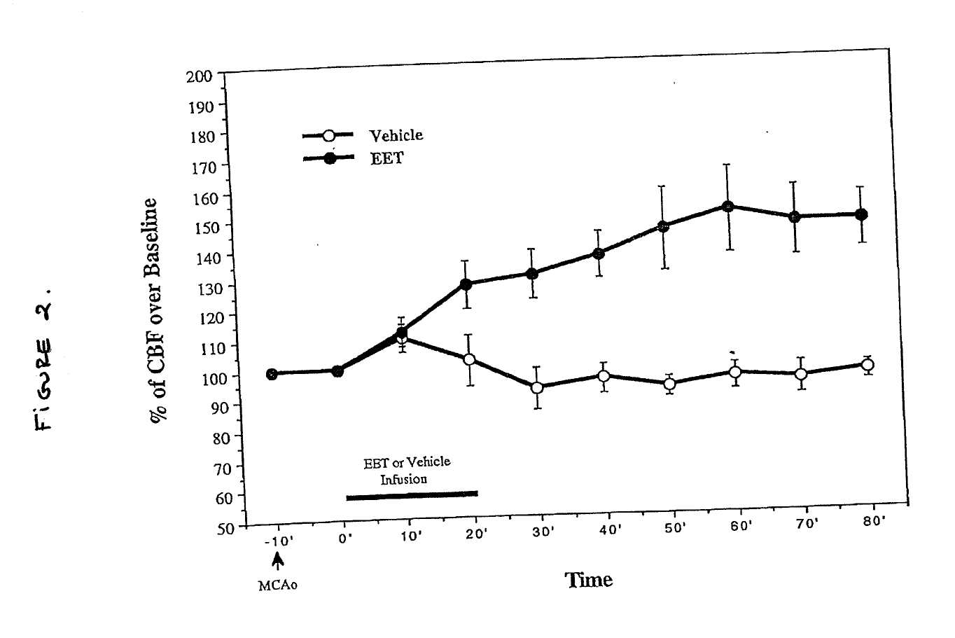

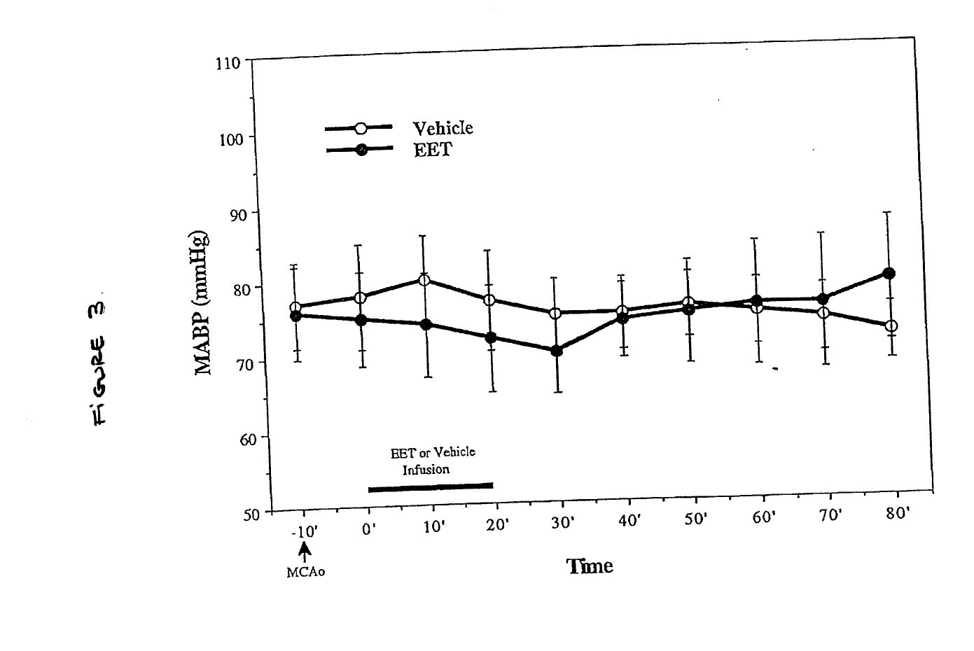

Physiological variables during laser-Doppler flowimetry in anesthetized, ventilated, EET-treated and vehicle-treated mice are shown in FIGS. 2 and 3. Values are reported as mean +/− SEM. CBF denotes cerebral blood flow; MABP indicates mean arterial blood pressure.

FIG. 2 is a graph showing regional CBF changes in EET-treated and vehicle-treated mice for 80 min after EET (300 μg/kg total dose) or vehicle infusion at a constant rate of 100 μl/kg/min over 20 min. Error bars denote standard error of the mean (SEM); CBF change with baseline control was determined by one-way ANOVA followed by Fisher's protected least-squares difference test. EET infusion (300 μg/kg, i.v.) increased rCBF in parietal cortex in mice, as shown in FIG. 2. The increase in rCBF began at 5-10 minutes and achieved statistical significance at 10-15 minutes after infusion. Maximum values achieved at 40-60 min reached 50% above baseline levels. By contrast, vehicle infusion (saline) did not increase rCBF.

FIG. 3 is a graph showing the MABP of EET-treated vs. Vehicle-treated animals. No significant changes in the MABP between the two groups of treated animals were realized.

TABLE 1
Infarct Volume (mm³) in mice subjected to middle cerebral
artery occlusion

| | | | EET | |
|---|---|---|---|---|
| | Vehicle | 30 μg/kg | 100 μg/kg | 300 μg/kg |
| | 36.3 | 27.7 | 24.5 | 11.8 |
| | 28.9 | 37.4 | 38.6 | 25.6 |
| | 28.0 | 23.7 | 11.8 | 13.6 |
| | 42.0 | 32.4 | 34.1 | 32.5 |
| | 41.8 | 43.0 | 37.6 | 13.4 |
| | 22.0 | 41.9 | 22.4 | 13.1 |
| | 40.2 | | 16.3 | 28.3 |
| Mean | 34.2 | 34.3 | 26.5 | 19.7 |
| S.D. | 7.9 | 7.8 | 10.5 | 8.7 |
| S.E.M. | 3.0 | 3.2 | 4.0 | 3.3 |
| n | 7.0 | 6.0 | 7.0 | 7.0 |

In Vitro Cell Culture Model

To test the effects of EET during oxidative stress, rat cerebellar granule cell (CGC) cultures were subjected to H₂O₂ treatment (225 μM for 10 min.). Less apoptosis was found at 24 hour when 11,12-EET (1.2 μM) was present compared to cell culture without 11,12-EET (51.7% vs. 87.6% respectively, p<0.01, scored by Hoechst stain for nuclear condensation). In addition, superoxide production (as monitored by oxidation of dihydroethidine) was decreased when 11,12-EET was present in CGC cultures during H₂O₂ treatment.

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method for reducing brain injury resulting from a cerebrovascular condition in a subject, comprising:
administering to a subject having a cerebrovascular condition, an epoxyeicosatrienoic acid in an amount effective to reduce brain injury in the subject.

2. The method of claim 1, wherein the cerebrovascular condition is selected from the group consisting of stroke, subarachnoid hemorhage, traumatic brain injury, and CADACIL syndrome.

3. The method of claim 1, wherein the epoxyeicosatrienoic acid is selected from the group consisting of 5,6-epoxyeicosatrienoic acid, 8,9-epoxyeicosatrienoic acid, 11,12-epoxyeicosatrienoic acid, and 14,15-epoxyeicosatrienoic acid.

4. The method of claim 1, wherein the epoxyeicosatrienoic acid is 11,12-epoxyeicosatrienoic acid.

5. The method of claim 1, further comprising co-administering arachidonic acid, a cytochrome P-450 arachidonic acid epoxygenase inducer, an agent other than a epoxyeicosatrienoic acid useful in the treatment of a cerebrovascular condition, or a combination thereof.

6. A method for reducing brain injury resulting from a cerebrovascular condition in a subject, comprising:
administering to a subject having a cerebrovascular condition, a cytochrome P-450 arachidonic acid epoxygenase inducer in an amount effective to reduce brain injury in the subject.

7. The method of claim 1, wherein the cerebrovascular condition is selected from the group consisting of stroke, subarachnoid hemorhage, traumatic brain injury, and CADACIL syndrome.

8. The method of claim 6, wherein the cytochrome P-450 arachidonic acid epoxygenase inducer is selected from the group consisting of 2,3,7,8-tetrachlorodibenzo-p-dioxin, β-naphthoflavone, and 3-methylcholanthrene.

9. The method of claim 6, further comprising co-administering arachidonic acid, an epoxyeicosatrienoic acid, an agent other than a cytochrome P-450 arachidonic acid epoxygenase inducer useful in the treatment of a cerebrovascular condition, or a combination thereof.
